(19) United States
(12) Patent Application Publication
(10) Pub. No.: US 2020/0190153 A1
(43) Pub. Date: Jun. 18, 2020

(54) CONSTITUTIVELY ACTIVE PROFILIN-1 FOR USE IN THE THERAPY AND/OR TREATMENT OF A NEUROLOGICAL DISORDER AND/OR FOR PROMOTING NEURONAL REGENERATION, KIT AND PRODUCTS THEREOF

(71) Applicant: INSTITUTO DE BIOLOGIA MOLECULAR E CELULAR - IBMC, Porto (PT)

(72) Inventors: Monica Luisa RIBEIRO MENDES DE SOUSA, Porto (PT); Sérgio Ricardo CARVALHO LEITE, Porto (PT); Ana Rita PINTO COSTA, Porto (PT); Raquel ALBUQUERQUE SIMÕES BAETA MENDES, Porto (PT); Joana Beatriz ANTUNES MOREIRA CARVALHO MARQUES, Porto (PT); Sara Patrícia CASTRO SOUSA, Porto (PT)

(21) Appl. No.: 16/611,154

(22) PCT Filed: May 7, 2018
(86) PCT No.: PCT/IB2018/053158

§ 371 Date: Nov. 5, 2019

(30) Foreign Application Priority Data
May 5, 2017 (PT) 110059
Feb. 26, 2018 (PT) 110593

(51) Int. Cl. C07K 14/47; C12N 15/86; C12N 7/00; A61K 9/00

(52) U.S. Cl. CPC C07K 14/47; C12N 15/86; A61K 38/00; A61K 9/0019; C12N 7/00

(57) ABSTRACT

The present disclosure relates to constitutively active profilin-1 (Pfn1S137A) for use in the therapy and/or treatment of a neurological disorder and/or for promoting neuronal regeneration, kit and related products thereof.

### CROSS-REFERENCE TO RELATED PATENT APPLICATIONS

This application is a U.S. National Stage Application under 35 U.S.C. § 371 of International Patent Application No. PCT/1132018/053158, filed May 7, 2018, which claims the benefit of priority to Portuguese Patent Application No. PT 110059 filed May 5, 2017 and Portuguese Patent Application No. PT 110593 filed Feb. 26, 2018, all of which are hereby incorporated by reference as if set forth in their respective entireties herein.

### TECHNICAL FIELD

The present disclosure relates to the use of constitutively active profilin-1 (Pfn1S137A) for use in the therapy and/or treatment of a neurological disorder and/or for promoting neuronal regeneration, kit and related products thereof.

### GENERAL DESCRIPTION

Mammalian neurons readily extend their axons during embryonic development. Upon embryonic to adult transition, the intrinsic neuronal growth activity is repressed to allow for proper synaptic development such that adult neurons are in a non-regenerative status. As such, in the mature vertebrate central nervous system (CNS), axons mostly fail to spontaneously regenerate, posing a major obstacle in the treatment of neurological disorders and CNS injury. A key principle guiding research in axon regeneration is that extrinsic cues in the environment of neurons, as well as cell-intrinsic mechanisms, contribute to the limited capacity of neurons to extend axons in the diseased/injured CNS. While progress has been made in characterizing the extrinsic cues that inhibit axon growth, the cell-intrinsic mechanisms that govern axon growth and regeneration remain poorly understood. This inability to activate a pro-regenerative program is a key culprit for the failure of adult CNS axons to rebuild a competent growth cone and regenerate after injury.

Regardless of the general inability of CNS axons to regenerate, it is possible to stimulate the intrinsic growth capacity of specific CNS axons. In sensory dorsal root ganglia (DRG) neurons, when the peripheral axon is injured—a paradigm known as conditioning lesion—the central axon gains regenerative capacity and is capable of regrowing within the inhibitory spinal cord injury site. To identify the molecules underlying this effect, it was performed a proteomic comparison of DRG neurons collected from rats with SCI (non-regenerative condition) with those of rats where SCI was preceded by a priming sciatic nerve lesion-conditioning lesion (high-regenerative condition). The proteomic data now disclosed strongly supported a central role of profilin-1 (Pfn1) in axon growth and regeneration. Pfn1 provides the pool of competent ATP-actin monomers that can be added to free filamentous actin ends, such as those in the peripheral domain of the growth cone, to support their polymerization and dynamics.

An aspect of the present disclosure demonstrates that the levels and activity of profilin-1 are critical for actin and microtubule (MT) dynamics required for optimal axon growth and regeneration.

The present disclosure demonstrates the central role of profilin-1 (Pfn1) in supporting optimal axon growth and regeneration.

Using the conditioning lesion, a model in which the axon regeneration capacity of spinal dorsal column axons is increased following a priming lesion to the sciatic nerve, it was determined that the total levels of Pfn1 are increased in regenerating axons whereas the inactive form of the protein is significantly decreased. In vitro, overexpression of constitutively active Pfn1 (Pfn1S137A) strongly enhanced actin and MT dynamics, and neurite outgrowth.

The present disclosure shows that in vitro, the acute knockdown of Pfn1 severely impairs axon formation/growth in hippocampal neurons and axon growth in dorsal root ganglia (DRG) neurons. Interestingly, ablation of Pfn1 did not only reduce actin dynamics but it also significantly decreased microtubule growth speed. In vivo, mice with an inducible neuronal deletion of Pfn1 had decreased axon regeneration of both peripheral and central DRG axons, further supporting the key role of Pfn1 for optimal axon (re)growth.

In vivo, AAV-mediated delivery of constitutively active Pfn1 increases axon regeneration after sciatic nerve injury; its effect after spinal cord injury is currently being evaluated. In summary, the experimental data shows that Pfn1 is a determinant of axon regeneration capacity acting.

In an embodiment, profilin is a ubiquitous cytosolic protein being a key player in the dynamics of the actin cytoskeleton. Given profilin's role in this key component of all cell types, it is anticipated that dysregulation of its basal activity could result in a wide variety of diseases. In fact, Pfn1 has been related to several medical conditions including Amyotrophic Lateral Sclerosis (ALS), cancer (glioblastoma and breast cancer, among others), atherosclerosis and other vascular disorders. In this respect it is very important that strategies targeting Pfn1 activity in neurons are strictly cell-specific, to avoid secondary effects resulting from dysregulation of Pfn1 activity in other cell types.

### DESCRIPTION OF THE DRAWINGS

The following figures provide preferred embodiments for illustrating the description and should not be seen as limiting the scope of the disclosure.

FIGS. 1A-1E. Increased activity of Pfn1 is required for optimal axon regeneration. (FIG. 1A) Schematic representation of the conditioned spinal cord injury paradigm used in the work (Left of grey dashed line: non-conditioned spinal cord injury, SCi; Right of grey dashed line: conditioned spinal cord injury, CL). Samples for western blot (WB) analysis were obtained from the injury site (A-5) one week after spinal cord injury (A-1). (FIGS. 1B and 1C) WB analysis (1B) and quantification (1C) of p137Pfn1, Pfn1 and ROCK1 levels at the spinal cord injury site (A-5) from conditioned and non-conditioned rat spinal cords. p-value *<0.05. (FIGS. 1D, 1E) Total levels of Pfn1 are increased in fast-growing axons. (1D) Quantification of the ratio of total levels of Pfn1/βIII-tubulin in relation to the distance from the leading edge of the growth cone. p-value ****<0.0001. (1E) Representative immunofluorescence of Pfn1 and βIII-tubulin in growth cones of conditioned and non-conditioned DRG neurons. Scale bar: 10 μm.

FIGS. 2A-2L. The acute deletion of Pfn1 impairs neuritogenesis and neurite outgrowth. Pfn1 depleted neurons show impaired neurite extension and cytoskeleton defects. (FIGS. 2A-2D) Embryonic day 18 (E18) rat hippocampal neurons were co-nucleofected with a pMAX-GFP and a control-pLKO plasmid or a Pfn1 ShRNA-pLKO plasmid. βIII-tubulin immunofluorescence (2A) and axon (2B)/dendrite (2C) outgrowth quantifications are shown. p-value****<0.0001. Scale bar: 50 μm. (2D) Percentage of neurons at different developmental stages. (FIGS. 2E-2H) Actin retrograde flow (2E, 2F) and microtubule growth speed (2G, 2H) analysis using LifeAct-GFP or EB3-GFP transfections, respectively. p-value****<0.0001. (FIGS. 2I-2L) Adult (2I, 2J) and E16 (2K, 2L) dorsal root ganglia (DRG) neurons were co-nucleofected with a pMAX-GFP and a control-pLKO plasmid or a Pfn1 ShRNA-pLKO plasmid. Total neurite length quantifications (2I, 2K) and branching analysis (2J, 2L) are shown. p-value****<0.01.

FIGS. 3A-3I. Profilin-1 is required for optimal axon growth in vitro. (FIG. 3A-3D) Demonstration of Pfn1 depletion in brain (3A-3C) of Cre+Pfn1wt/wt (control) and Cre+Pfn1fl/fl (with specific inducible neuronal deletion of Pfn1) mice. No changes in Pfn2 levels in this samples (3A, 3C). (FIGS. 3E-FG) Neurite outgrowth assay of Cre+Pfn1wt/wt and Cre+Pfn1fl/fl DRG neurons either transfected with a control-pLKO plasmid or with a Pfn2 ShRNA-pLKO. p-value ****<0.0001. Representative βIII tubulin immunofluorescence (3E), total neurite length (3F) and mean number of branches (3G) are shown. Scale bars: 50 μm. p-value ***<0.001. (FIGS. 3H, 3I) Actin retrograde flow (3H) and microtubule growth speed (3I) analysis in growth cones of Cre+Pfn1wt/wt, Cre+Pfn1fl/fl DRG neurons using LifeAct-RFP and EB3-mCherry transfections, respectively. p-value ***<0.001,**<0.01 and *<0.05.

FIGS. 4A-4F. Profilin-1 is required for optimal axon regeneration in vivo; peripheral nervous system (PNS) and central nervous system (CNS) regeneration analysis. (FIG. 4A) Cre+Pfn1wt/wt YFP sciatic nerve section. (FIG. 4B) Representative images of PPD-stained semithin sciatic nerve sections from Cre+Pfn1wt/wt and Cre+Pfn1fl/fl mice 2 weeks after sciatic nerve (SN) crush; scale bar: 50 μm. (FIG. 4C) Quantification of myelinated axon density illustrated in (FIG. 4B). Error bars are SEM. p-value **<0.005. (FIG. 4D) Representative images of cholera toxin B-positive (CT-B+) fibers in sagittal spinal cord sections following conditioning lesion (CL) in Cre+Pfn1wt/wt and Cre+Pfn1fl/fl mice. YFP+ axons are shown in green and dorsal column fibers traced with CT-B are labeled in red. The double positive YFP+/CT-B+ axons are highlighted with arrows; scale bar: 100 μm; dashed lines label the border of the glial scar. (FIG. 4E) Quantification of the number of CT-B+/YFP+ dorsal column fibers that are able to enter in the glial scar. (FIG. 4F) Quantification of the length of the regenerating axons within the glial scar, from the lesion border. All error bars are SEM. p-value *<0.05.

FIGS. 5A-5K. Increased Pfn1 activity is crucial for optimal axon growth. Adult DRG neurons (FIGS. 5A-5E) and E16.5 mice hippocampal neurons (FIGS. 5F-5J) were co-transfected with pMAX-GFP and WT or Pfn1S137A plasmid; the overexpression of the WT and Pfn1S137A was confirmed in CAD cell extracts (FIG. 5K). Representative βIII tubulin immunofluorescences of adult DRG (5A, scale bar: 200 μm) and DIV4 hippocampal neurons are shown (5F, scale bar: 100 μm). Quantification of total neurite length (5B) and branching analysis (5C) for DRG neuron cultures and axonal (5G) and dendritic (5H) outgrowth for DIV4 hippocampal neurons are shown. Actin retrograde flow (5D-DRG neurons; 5I-hippocampal neurons) and microtubule growth speed (5E-DRG neurons; 5J-hippocampal neurons) were quantified. p-value *<0.05, **<0.01, ****<0.0001.

FIG. 6. In vivo, AAV-mediated delivery of constitutively active Pfn1 increases axon regeneration after sciatic nerve injury. Quantification of the length of regenerating axons distally to the sciatic nerve injury boarder after delivery of either control AAV or AAV carrying constitutively active Pfn1. p-value *<0.05.

### DETAILED DESCRIPTION

The present disclosure relates to the use of constitutively active profilin-1 (Pfn1S137A) for use in the therapy and/or treatment of a neurological disorder and/or for promoting neuronal regeneration, kit and related products thereof.

In the present disclosure the constitutively active profilin-1 means profilin-1 where by site-directed mutagenesis the residue Serine 137 was replaced by an Alanine (Pfn1S137A). Profilin-1 is inactivated by phosphorylation in Serine 137; if this residue is replaced by an Alanine, that cannot be phosphorylated, the protein becomes constitutively active.

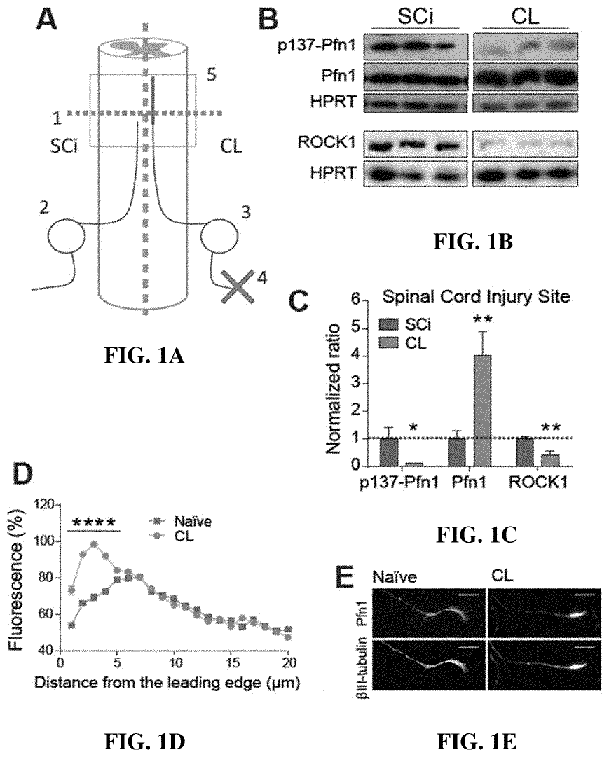

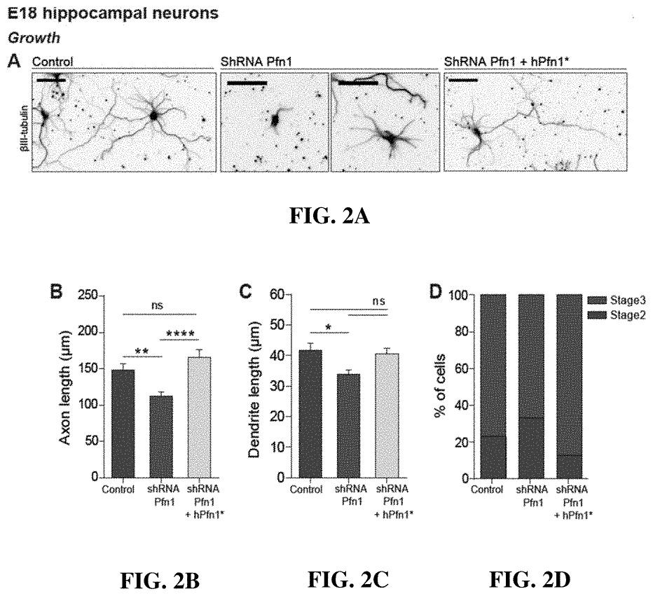

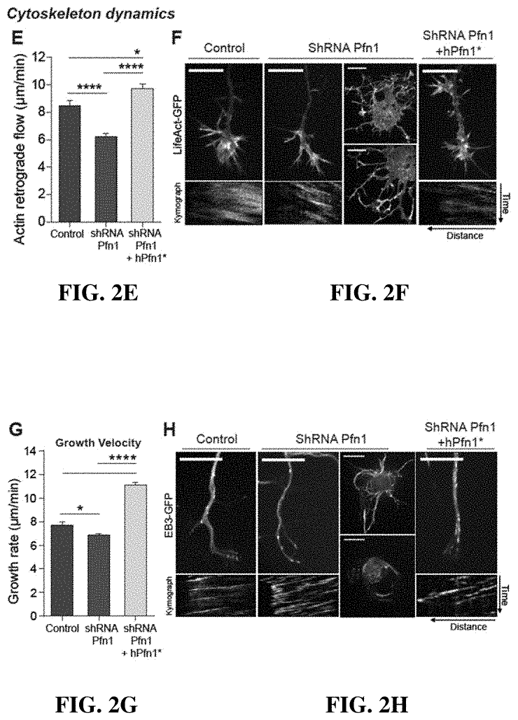

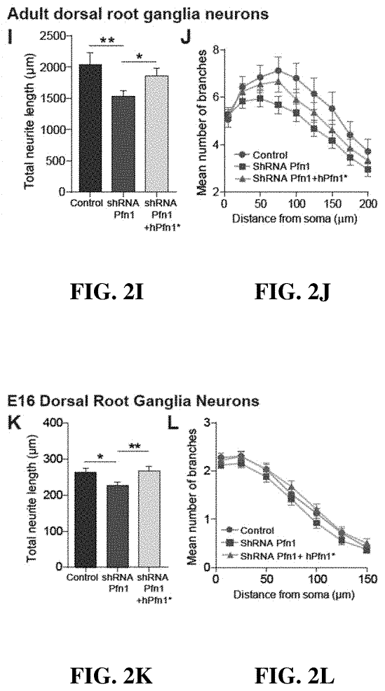

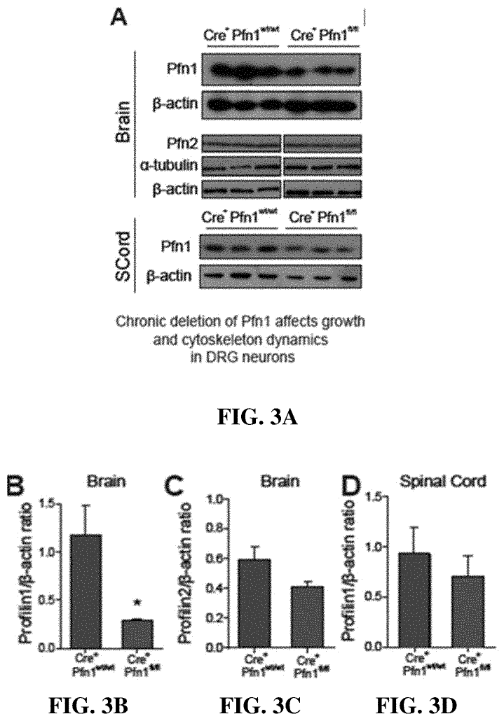

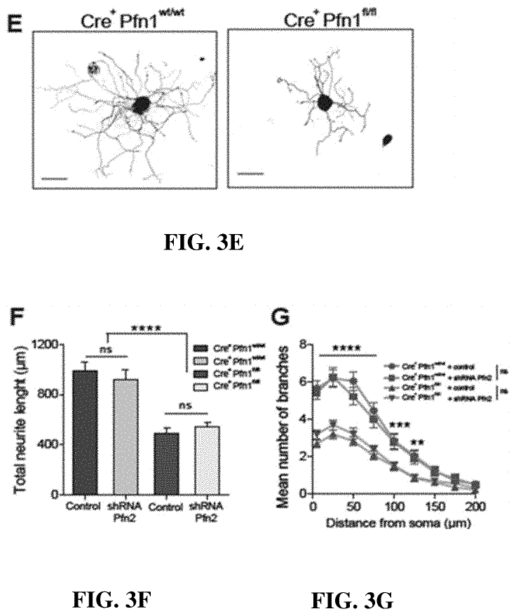

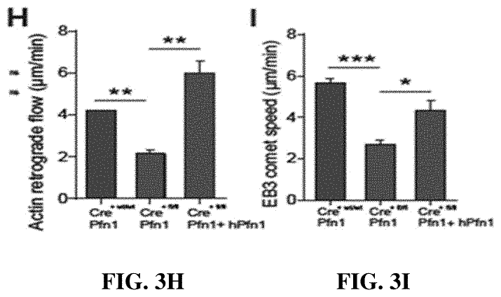

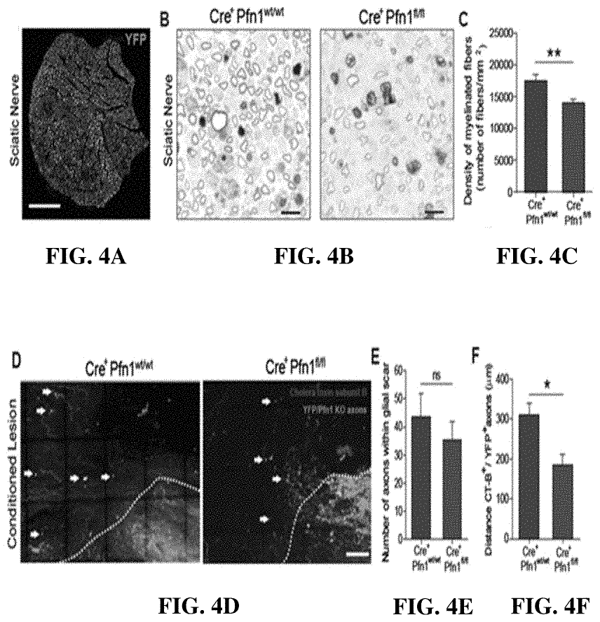

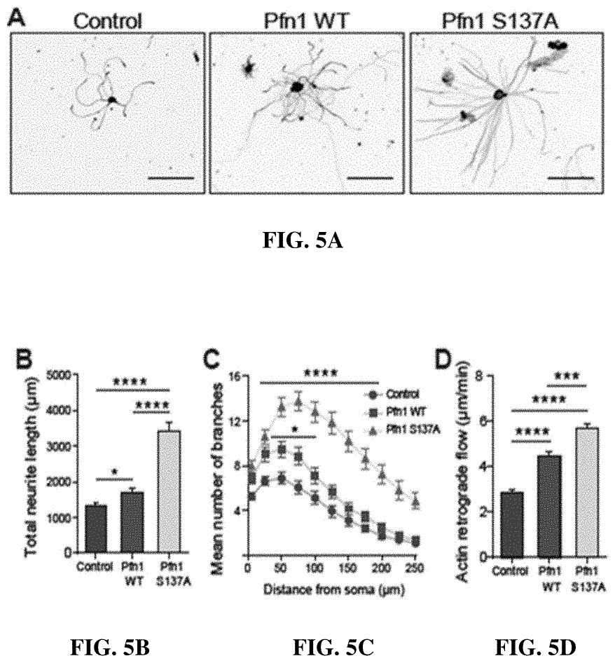

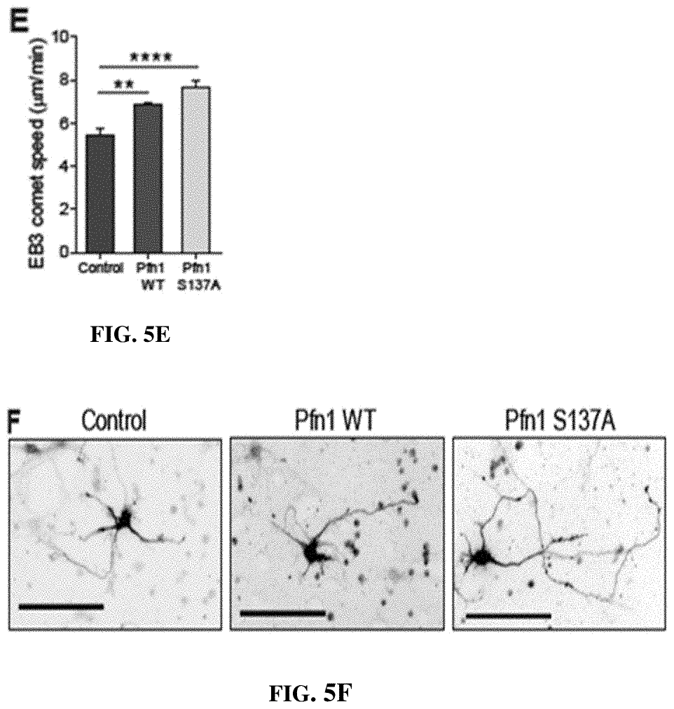

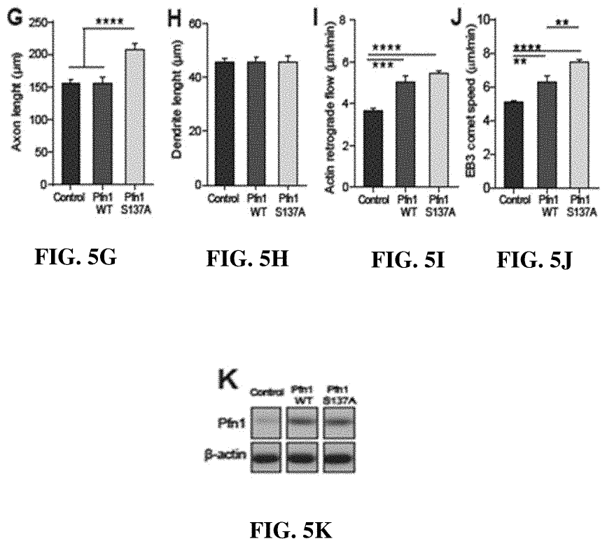

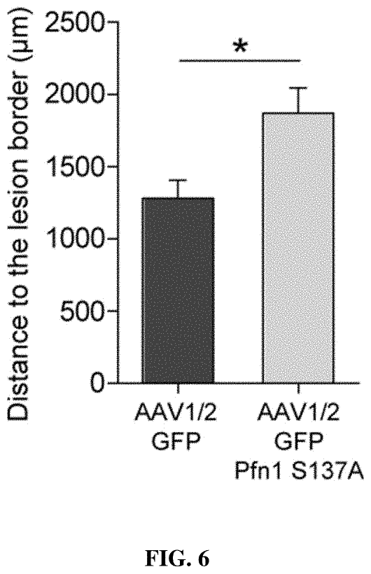

In an embodiment FIGS. 1A-1E illustrate that the activity of Pfn1 is required for optimal axon regeneration.

An aspect of the present disclosure relates to a constitutively active profilin-1, i.e. Pfn1 in which the residue Ser137 was mutated into an Ala to generate a phospho-resistant form of the protein, Pfn1-Pfn1S137A, for use in the therapy and/or treatment of a neurological disorder and/or for promoting axon regeneration, In an embodiment, the present disclosure relates to constitutively active profilin-1 for use in the treatment or therapy of central and/or peripheral nervous system injury or disorder.

In an embodiment, the present disclosure relates to a constitutively active profilin-1 for use in the therapy and/or treatment of a neurological disorder, selected from the group consisting of peripheral neuropathies cause by physical injury or disease state, physical damage to the brain, physical damage to the spinal cord, stroke associated with brain damage, and neurological disorders related to neurodegeneration.

In an embodiment, the present disclosure relates to a constitutively active profilin-1 for use in the therapy and/or treatment of a neurological disorder selected from the group consisting of neuralgias, muscular dystrophy, bell's palsy, myasthenia gravis, Parkinson's disease, Alzheimer's disease, multiple sclerosis, stroke and ischemia associated with stroke, neural neuropathy, other neural degenerative disease, motor neuron disease, and nerve injury. In particular, wherein the injured nerve tissue is spinal cord tissue.

In an embodiment, the injured nerve tissue is peripheral nerve tissue.

In an embodiment, the injury is selected from the group consisting of a mechanical injury, a biochemical injury and an ischemic injury.

Another aspect of the present disclosure relates to a gene construct comprising constitutively active profilin-1, in particular Pfn1S137A, described in the present disclosure.

Another aspect of the present disclosure relates to a vector comprising the gene construct encoding the constitutively active profilin-1, in particular Pfn1S137A, of the present disclosure.

In an embodiment, the vector is a viral vector.

In an embodiment, the viral vector is capable to target neurons.

In an embodiment, the viral vector is a recombinant adeno-associated virus, in particular wherein the recombinant adeno-associated virus is of a serotype selected from the group consisting of AAV1, AAV2, AAV3, AAV4, AAV5, AAV6, AAV7, AAV8, AAV9, AAV10, AAV11, AAV12, and hybrids thereof.

Another aspect of the present disclosure relates to a pharmaceutical composition comprising an effective amount of constitutively active profilin-1 (Pfn1S137A) or of the vector, described in the present disclosure and a suitable carrier.

In an embodiment, the pharmaceutical composition is an injectable formulation, in particular an in situ or systemic injection.

In an embodiment, the minimum concentration of the vector is 10¹² genome copies/ml (GC/ml).

Another aspect of the present disclosure relates to a kit comprising the constitutively active profilin-1 described in the present subject matter, the pharmaceutical composition or the vector described in the present disclosure.

In an embodiment, the enhanced green fluorescent protein (eGFP) linked to the self-cleaving small peptide 2A, linked to profilin-1 Ser137Ala (Pfn1S137A), was cloned into an adeno-associated virus 1 (AAV1) plasmid driven by the cytomegalovirus (CMV) promoter (AAVLCMV.PLeGFP.WPRE.bGH) to obtain the construct AAV1.CMV.eGFP-T2A-Pfn1S137A.WPRE.bGH. Control AAV vector, where Pfn1S137A is replaced by a 5Gly sequence was also be generated (AAV1.5Gly-T2A-eGFP.WPRE.bGH). The AAV vectors were produced as described in Lock M, Alvira M, Vandenberghe L H, Samanta A, Toelen J, Debyser Z, Wilson J M. 2010. Rapid, simple, and versatile manufacturing of recombinant adeno-associated viral vectors at scale. Hum Gene Ther. 21:1259-1271. Both vectors were packaged in AAV2/1 particles (with AAV1 viral capsid and with AAV2 inverted terminal repeats). Genome copy (GC) titers of AAV vectors were determined. For sciatic nerve injury (SNI), 2 μL (minimum 10¹² GC/ml) of either control or experimental AAVs were injected in each L4 and L5 DRGs using a Hamilton syringe (33G) (n=8 rats/group). One week later the rat sciatic nerves were crushed at the level of the sciatic notch and 3 days later, sciatic nerve distal to the lesion site was collected to analysis of axon regeneration. Following sciatic nerve injury, constitutively active Pfn1 delivery induced a 1.5-fold increase in the distance that axons regenerated distally to the injury boarder. For spinal cord injury (SCI), ascending dorsal column axons were traced by injecting 2 μL (minimum 10¹² GC/ml) of either control or experimental AAVs into the left sciatic nerve of 12-week old Wistar rats using a Hamilton syringe (33G) (n=8 rats/group). Two weeks later a laminectomy was performed at the T9-T10 level and the dorsal half of the spinal cord was cut using a micro feather ophthalmic scalpel. Functional analysis of the animals was performed weekly after injury using the BBB score and the Von Frey filaments test. Rats were allowed to recover for 6 weeks before collecting the injured spinal cords for the analysis of regenerating eGFP-positive axons. Specifically, rats were transcardially perfused with 4% paraformaldehyde and the spinal cords were post-fixed for 1 week and later transferred to 30% sucrose in PBS before tissue processing. Serial cryosections (50 μm thick) of the spinal cord were cut in the sagittal plane and immunofluorescence against SCG10/Stathmin-2 (1:5000, NBP1-49461 Novus Biolologicals) was done to identify regenerating sensory axons. Regenerating axons were traced rostrally to the injury site (2000 μm rostral to the lesion boarder). Following spinal cord injury, constitutively active Pfn1 delivery induced a 1.4-fold increase in the distance that axons regenerated distally to the injury boarder.

In summary, the data shows that vitro, Pfn1 knockdown severely impaired actin retrograde flow, microtubule growth speed, and axon formation and growth. In vivo, mice with an inducible neuronal deletion of Pfn1 had decreased axon regeneration. In a model of high regeneration capacity, Pfn1 activity was increased in the growth cone of regenerating axons. In line with these findings, overexpression of constitutively active Pfn1 strongly enhanced actin and MT dynamics, and axon growth in vitro. In vivo, delivery of constitutively active Pfn1 increased axon regeneration following sciatic nerve injury and spinal cord injury. Overall, it is shown that Pfn1 is a determinant of axon growth and regeneration acting as a key regulator of both actin and MT dynamics in the growth cone.

The term “comprising” whenever used in this document is intended to indicate the presence of stated features, integers, steps, components, but not to preclude the presence or addition of one or more other features, integers, steps, components or groups thereof.

Where singular forms of elements or features are used in the specification of the claims, the plural form is also included, and vice versa, if not specifically excluded. For example, the term “a gene” or “the gene” also includes the plural forms “genes” or “the genes,” and vice versa. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or all of the group members are present in, employed in, or otherwise relevant to a given product or process.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the disclosure, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

1. A method of treating a neurological disorder, a nerve injury, and/or for promoting neuronal regeneration, in particular axon regeneration, in a patient, the method comprising:
administering an effective amount of constitutively active profilin-1 (Pfn1) to the patient.

2. The method of claim 1, wherein the profilin-1 (Pfn1) is Pfn1S137A.

3. The method of claim 1, wherein the neurological disorder is a central and/or peripheral nervous system injury or disorder.

4. The method of claim 1, wherein the neurological disorder is selected from the group consisting of peripheral neuropathies caused by physical injury or disease state, physical damage to the brain, physical damage to the spinal cord, stroke associated with brain damage, and neurological disorders related to neurodegeneration.

5. The method of claim 1, wherein the neurological disorder is selected from the group consisting of neuralgias, muscular dystrophy, bell's palsy, myasthenia gravis, Parkinson's disease, Alzheimer's disease, multiple sclerosis, stroke and ischemia associated with stroke, neural neuropathy, other neural degenerative disease, motor neuron disease, or nerve injury.

6. The method of claim 1, wherein the nerve injury involves injured nerve tissue and wherein the injured nerve tissue is spinal cord tissue.

7. The method of claim 1, wherein the nerve injury involves injured nerve tissue and wherein the injured nerve tissue is peripheral nerve tissue.

8. The method of claim 1, wherein the nerve injury is selected from the group consisting of a mechanical injury, a biochemical injury and an ischemic injury.

9. (canceled)

10. A vector comprising a constitutively active profilin-1 (Pfn1), wherein the profilin-1 (Pfn1) is Pfn1S137A.

11. The vector of claim 10, wherein the vector is a viral vector.

12. The vector of claim 11, wherein the viral vector is capable of targeting a neuron.

13. The vector of claim 11, wherein the viral vector is a recombinant adeno-associated virus, in particular wherein the recombinant adeno-associated virus is of a serotype selected from the group consisting of AAV1, AAV2, AAV3, AAV4, AAV5, AAV6, AAV7, AAV8, AAV9, AAV10, AAV11, AAV12, and hybrids thereof.

14. A pharmaceutical composition comprising a suitable carrier and an effective amount of a constitutively active profilin-1 Pfn1S137A, or a vector of the constitutively active profilin-1 Pfn1S137A.

15. The pharmaceutical composition of claim 14, wherein the composition is an injectable formulation, in particular an in situ or systemic injectable formulation.

16. The pharmaceutical composition of claim 14, wherein the minimum concentration of the vector is 10¹² GC/ml.

17. (canceled)
